(19) United States
(12) Patent Application Publication
(10) Pub. No.: US 2010/0316711 A1
(43) Pub. Date: Dec. 16, 2010

(54) NIFEDIPINE CONTAINING OPRESS COATED TABLET AND METHOD OF PREPARING SAME

(75) Inventors: Yuka Yamanouchi, Osaka (JP); Kosuke Fukui, Shiga (JP)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: BAYER YAKUHIN ,LTD., Osaka-shi ,Osaka (JP)

(21) Appl. No.: 12/739,467

(22) PCT Filed: Oct. 24, 2008
(86) PCT No.: PCT/JP2008/069828

§ 371 Date: Sep. 2, 2010

(30) Foreign Application Priority Data
Oct. 25, 2007 (JP) 2007-277908

(51) Int. Cl. A61K 9/36; A61K 31/4422; A61P 9/12

(52) U.S. Cl. 424/469; 514/356

(57) ABSTRACT

A sustained release nifedipine-containing press coated tablet, which comprises (A) a core comprising nifedipine and a hydrophilic gel-forming high molecular substance, wherein the dissolution of the nifedipine from the core is designed to be delayed: and (B) an outer shell coating said core, which comprises (i) (a) nifedipine and (b) a disintegration suppression substance comprising a pH-independent water-insoluble polymer, wherein the combined contents of (a) and (b) are in the range of 8 to 22% by weight based on the total weight of the outer shell, and (ii) a hydrophilic gel-forming high molecular substance selected from cellulose derivatives and polyvinyl alcohols, wherein the content thereof is in the range of 76 to 95% by weight based on the total weight of the outer shell, which is characterized in that the diameter of said tablet is 7.5 to 8.5 mm, and the thickness thereof is 4.5 to 5.2 mm, and further characterized in that the dissolution rate of nifedipine from said tablet is controlled within a specific range. The tablet of the present invention is miniaturized as compared to the currently-used nifedipine-containing tablets but the dissolution properties and bioavailability of nifedipine therein are not changed.

### TECHNICAL FIELD

The present invention relates to a nifedipine-containing press coated tablet and a method of preparing the same. More particularly, the present invention relates to a miniaturized nifedipine-containing press coated tablet for once-a-day administration, and a method of preparing the same, and further relates to a combination drug of said tablet and a second pharmaceutically active ingredient.

### BACKGROUND ART

The present inventors have developed before a nifedipine-containing press coated tablet for once-a-day administration, and disclosed it in Patent Document 1. Said nifedipine-containing press coated tablet (trade name: Adalat CR) has been clinically and commonly administered to hypertensive patients in Japan. However, said press coated 10 mg tablet and 40 mg tablet are rather large in size (i.e., diameter: about 9 mm, thickness: about 5.5 mm, volume: about 310 mm³), that is an inherent feature in press coated tablets. Therefore, it is hardly said that said press coated tablets are easy for elder patients or patients having a swallowing problem to take. Under these circumstances, it has been desired to miniaturize these tablets.

In addition, it is speculated that about half of hypertensive patients taking said press coated tablets may additionally take other antihypertensive agent. In fact, according to the statistics of IMS (2001), the percentage of patients taking Adalat CR together with an angiotensin converting enzyme inhibitor such as enalapril, lisinopril, etc. is about 15%, and the percentage of patients taking Adalat CR together with an angiotension II receptor antagonist such as candesartan, valsartan, etc. is about 20%. The reasons why the percentage of taking both of Adalat CR and other antihypertensive agent is so high are (1) there are a quite lot of patients who cannot obtain sufficient antihypertensive effects and effects of sustaining blood pressure by monotherapy of each antihypertensive agent; (2) in order to obtain sufficient effects by monotherapy of each antihypertensive agent, the dosage thereof needs to be increased, by which the risk of onset of side effect is also increased; (3) further, the combination therapy using plural antihypertensive agents having different action mechanisms in a low dose is considered to be safer, etc.

In these combination therapies, at least one of other agents is administered in addition to the above mentioned nifedipine-containing press coated tablet having a rather large size, by which the burden on the patients is increased. Therefore, the miniaturization of said nifedipine-containing press coated tablet is especially needed in the combination therapy. Further, if said nifedipine-containing, once-a-day administration type, press coated tablet can be miniaturized enough, then it may be possible to prepare a combination drug of nifedipine and other second pharmaceutically active ingredient as mentioned above in a single dosage form, by which it is expected to reduce the physical burden on the patients.

The size of the final tablet being desirable for the patients can be estimated such as about 7 to 8 mm of diameter, about 4 to 5 mm of thickness, and further in order to secure the efficiency and safety of nifedipine preparation for once-a-day administration even after miniaturization, the dissolution profile of nifedipine therefrom should not be different from that of nifedipine from the commercially available nifedipine press coated tablet but rather the dissolution profile of nifedipine from the commercially available nifedipine press coated tablet should be maintained.

Patent Document 1 discloses a nifedipine-containing press coated tablet, but said tablet is not one where the miniaturization thereof is aimed. In addition, Patent Document 2 discloses a technique of miniaturizing nifedipine preparation, that is, it discloses a technique where a disintegration suppression substance is blended into a core of nifedipine-containing press coated tablet by which the core is miniaturized, and by miniaturization of the core, the dissolution profile of nifedipine from the final tablet is secured. However, the present invention is different from the technique of Patent Document 2 in that the core of the present tablet does not contain any disintegration suppression substance and the diameter and the thickness of the core do not make much difference from those of conventional Adalat CR, and further in that the desired nifedipine dissolution profile and bioavailability in the final tablet can be secured by miniaturizing only the outer shell of the present tablet.

Especially, in order to miniaturize the final tablet, it is more desirable to miniaturize the outer shell having a larger volume than to miniaturize the core having a smaller volume from the viewpoint that both of suitable nifedipine dissolution profile and bioavailability are secured even after miniaturization of the final tablet, because when the nifedipine content per nifedipine-containing press coated tablet is increased, the nifedipine contents in the core and the outer shell are increased so that excipients contained therein must be reduced, but it is necessary to add the excipients in a certain amount at a minimum in order to secure both suitable nifedipine dissolution profile and bioavailability.

When miniaturizing the outer shell, it was thought to suppress the dissolution of nifedipine by combining hydrophilic gel-forming polymers having various viscosity levels. However, it was difficult to secure the dissolution profile and bioavailability of nifedipine in the final tablet by simply adjusting the hydrophilic gel-forming high molecular substances, because of the decrease in the surface area of tablet due to miniaturization, the change of the mechanical strength of tablet, or the decrease in the amount of excipients to be blended.

[Patent Document 1] U.S. Pat. No. 3,220,373

[Patent Document 2] U.S. Pat. No. 3,751,287

### DISCLOSURE OF INVENTION

The present invention provides a sustained release nifedipine-containing tablet, which is miniaturized in order to make easy for patients to take, and having the similar dissolution profile and bioavailability of nifedipine to those of the conventional nifedipine tablets.

The present inventors have intensively studied a method of miniaturization of the nifedipine-containing press coated tablets for once-a-day administration disclosed in Patent Document 1, said method being different from the method disclosed in Patent Document 2, and they have succeeded in obtaining a nifedipine-containing press coated tablet for once-a-day administration, of which volume is reduced to about 70% of the conventional press coated tablets, but still maintaining the nifedipine dissolution profile and bioavailability of the commercially available nifedipine-containing preparation Adalat CR, by keeping combined contents of nifedipine and a disintegration suppression substance in the outer shell constant instead of decreasing the contents of hydrophilic gel-forming high molecular substance and the disintegration suppression substance in the outer shell, and finally they have accomplished the present invention.

The present invention contains the following embodiments.

[1] A nifedipine-containing press coated tablet which comprises

(A) a core comprising nifedipine and a hydrophilic gel-forming high molecular substance, wherein the dissolution of the nifedipine from the core is designed to be delayed: and

(B) an outer shell which compressively coats the above core and which comprises

(i) (a) nifedipine and (b) a disintegration suppression substance comprising a pH-independent water-insoluble polymer, wherein the combined contents of (a) and (b) are in the range of 8 to 22% by weight based on the total weight of the outer shell, and

(ii) a hydrophilic gel-forming high molecular substance selected from cellulose derivatives and polyvinyl alcohols, wherein the content thereof is in the range of 76 to 95% by weight based on the total weight of the outer shell,

which is characterized in that in the outer shell, the disintegration suppression substance forms a matrix with nifedipine and a hydrophilic gel-forming high molecular substance, and the diameter of the nifedipine-containing press coated tablet is 7.5 to 8.5 mm, and the thickness thereof is 4.5 to 5.2 mm,
and further characterized in that the dissolution rate of the nifedipine from said tablet is:

(a) in the dissolution test using a sinker according to Method 2 of the dissolution test method as prescribed by The Japanese Pharmacopoeia,

10-40% after 2 hours;

30-65% after 4 hours;

at least 55% after 6 hours, and

(b) in the dissolution test according to the disintegration test method as prescribed by The Japanese Pharmacopoeia,

35-65% after 3 hours;

at least 65% after 4 hours.

[2] The nifedipine-containing press coated tablet according to the above [1], wherein the disintegration suppression substance is ethyl cellulose or ethyl acrylate•methyl methacrylated•trimethylammonium-ethyl methacrylate chloride copolymer.
[3] The nifedipine-containing press coated tablet according to the above [1], wherein the disintegration suppression substance is ethyl acrylate•methyl methacrylate•trimethylammoniumethyl methacrylate chloride copolymer.
[4] The nifedipine-containing press coated tablet according to any one of the above [1] to [3], wherein the hydrophilic gel-forming high molecular substance in the core and the outer shell is hydroxypropyl cellulose or hydroxypropylmethyl cellulose.
[5] The nifedipine-containing press coated tablet according to any one of the above [1] to [3], wherein the hydrophilic gel-forming high molecular substance in the outer shell is a combination of a medium viscosity hydroxypropyl cellulose and a low viscosity hydroxypropyl cellulose.
[6] The nifedipine-containing press coated tablet according to any one of the above [1] to [5], wherein the content ratio of the nifedipine and the disintegration suppression substance in the outer shell is 2:1 to 3:1.
[7] The nifedipine-containing press coated tablet according to any one of the above [1] to [5], wherein the content ratio of the nifedipine and the disintegration suppression substance in the outer shell is 1:1 to 1:4.
[8] The nifedipine-containing press coated tablet according to any one of the above [1] to [7], wherein the content of nifedipine in the outer shell is 3 to 15% by weight based on the total weight of the outer shell.
[9] The nifedipine-containing press coated tablet according to any one of the above [1] to [8], wherein the combined content of the nifedipine and the disintegration suppression substance in the outer shell is 18 to 21% by weight based on the total weight of the outer shell.
[10] The nifedipine-containing press coated tablet according to any one of the above [1] to [9], wherein the dissolution rate of the nifedipine from said tablet is:

40 to 60% after 3 hours;

65 to 90% after 4 hours,

in the dissolution test according to the disintegration test method as prescribed by The Japanese Pharmacopoeia.
[11] The nifedipine-containing press coated tablet according to any one of the above [1] to [10], wherein the diameter of the core is 4.5 to 5.5 mm, and the thickness thereof is 1.5 to 2.5 mm.
[12] A tablet, which is characterized by being prepared by partially compressively coating a nifedipine-containing press coated tablet as set forth in any one of the above [1] to [11] with a rapidly-dissolving part comprising a second pharmaceutically active ingredient in such a manner that at least a part of the surface of said press coated tablet is exposed.
[13] A method of preparing a nifedipine-containing press coated tablet, which comprises compressively coating (A) a core comprising nifedipine and a hydrophilic gel-forming high molecular substance, wherein the dissolution of the nifedipine from the core is designed to be delayed, with (B) an outer shell comprising (i) (a) nifedipine and (b) a disintegration suppression substance comprising a pH-independent water-insoluble polymer, wherein the combined contents of (a) and (b) are in the range of 8 to 22% by weight based on the total weight of the outer shell, and (ii) a hydrophilic gel-forming high molecular substance selected from cellulose derivatives and polyvinyl alcohols, wherein the content thereof is in the range of 76 to 95% by weight based on the total weight of the outer shell,
said tablet being characterized in that in the outer shell, the disintegration suppression substance forms a matrix with nifedipine and a hydrophilic gel-forming high molecular substance, and the diameter of the nifedipine-containing press coated tablet is 7.5 to 8.5 mm, and the thickness thereof is 4.5 to 5.2 mm,
and further characterized in that the dissolution rate of the nifedipine from said tablet is:
(a) in the dissolution test using a sinker according to Method 2 of the dissolution test method as prescribed by The Japanese Pharmacopoeia,

10-40% after 2 hours;

30-65% after 4 hours;

at least 55% after 6 hours, and

(b) in the dissolution test according to the disintegration test method as prescribed by The Japanese Pharmacopoeia,

35-65% after 3 hours;

at least 65% after 4 hours.

[14] The method of preparing the nifedipine-containing press coated tablet according to the above [13], wherein the diameter of the core is 4.5 to 5.5 mm, and the thickness thereof is 1.5 to 2.5 mm.
[15] A film-coating tablet, which comprises the tablet as set forth in any one of the above [1] to [12], and at least one layer of film coating for light protection being applied thereon.
[16] A method of preparing a film-coating tablet, which comprises formulating a nifedipine-containing press coated tablet by the method as set forth in the above [13] or [14], followed by applying at least one layer of film coating for light protection on the surface of said tablet.

The present invention is characterized by keeping the combined contents of the nifedipine and the disintegration inhibiting substance in the outer shell constant.

### BRIEF DESCRIPTION OF DRAWINGS

FIG. 1 shows the dissolution patterns of nifedipine from the tablets of Example 1 and Comparative Example 1, and the commercially available Adalat CR 40 mg, in the dissolution test using a sinker according to Method 2 of the dissolution test method as prescribed by the Japanese Pharmacopoeia [Pharmacopoeia Method 2 dissolution test (using sinker)].

FIG. 2 shows the dissolution patterns of nifedipine from the tablets of Example 1 and Comparative Example 1, and the commercially available Adalat CR 40 mg, in the dissolution test according to the disintegration test method as prescribed by the Japanese Pharmacopoeia (Pharmacopoeia Disintegration test device method).

FIG. 3 shows the dissolution patterns of nifedipine from the tablets (which were not applied with film coating for light protection) of Example 2, Example 3 and Comparative Example 2, and the commercially available Adalat CR 10 mg, in the dissolution test using a sinker according to Method 2 of the dissolution test method as prescribed by the Japanese Pharmacopoeia [Pharmacopoeia Method 2 dissolution test (using sinker)].

FIG. 4 shows the dissolution patterns of nifedipine from the tablets (which were not applied with film coating for light protection) of Example 2, Example 3 and Comparative Example 2, and the commercially available Adalat CR 10 mg, in the dissolution test according to the disintegration test method as prescribed by the Japanese Pharmacopoeia (Pharmacopoeia Disintegration test device method).

### BEST MODE FOR CARRYING OUT THE INVENTION

The outer shell of the nifedipine press coated tablet of the present invention contains three components such as nifedipine, a hydrophilic gel-forming high molecular substance and a disintegration suppression substance.

The properties of nifedipine used in the core and the outer shell are not necessarily specified. Normally, however, nifedipine crystals which are micro-pulverized to such a level that their median diameter as measured by sedimentation method or laser diffraction scattering-type particle size distribution method is about 1-30 μm; or the specific surface area as measured by gas adsorption method is about 0.5-10 m²/g, are preferably used. Nifedipine which has been made amorphous or solid solution together with polyvinylpyrrolidone, etc. and pulverized or nifedipine once dissolved in an organic solvent and thereafter adsorbed onto a porous substance such as light anhydrous silicic acid, etc., may also be used.

The press coated tablet of the present invention can generally contain 10-120 mg, preferably 20-90 mg, more preferably 30 to 90 mg of nifedipine per tablet. The relative ratio of nifedipine contents in the core and the outer shell is not strictly specified, while generally the weight ratio of nifedipine content of the core/nifedipine content of the outer shell may be within the range of 1/7-3/1, especially in the range of 1/5 to 2/1.

As to the content ratio of nifedipine in the outer shell, the combined contents of nifedipine and the disintegration suppression substance in the outer shell is adjusted into the range of 8 to 22% by weight based on the total weight of the outer shell. Namely, when the content of nifedipine in the outer shell is increased, then the content of the disintegration suppression substance therein is decreased. The ratio of nifedipine and the disintegration suppression substance in the outer shell is, for example, within the range of 2:1 to 3:1, or within the range of 1:1 to 1:4, but it is not necessarily specified.

Under these conditions, the content ratio of nifedipine in the outer shell is preferably within the range of 3 to 15% by weight, more preferably within the range of 10 to 15% by weight, based on the total weight of the outer shell.

As to the hydrophilic gel-forming high molecular substance to be used in the core and the outer shell, cellulose derivatives such as methyl cellulose, hydroxypropyl cellulose (HPC), hydroxypropyl methyl cellulose (HPMC), carboxymethyl cellulose sodium, etc. or polyvinyl alcohols, etc. are exemplified. Among them, hydroxy-lower alkyl ether of cellulose, especially HPC or HPMC is preferable. In the above “lower alkyl”, the term “lower” means “having carbon atoms of not more than 6”. These hydrophilic gel-forming high molecular substances may be used alone or in a combination of two or more thereof. In addition, hydrophilic gel-forming high molecular substances of various different viscosity levels may be used in a combination of two or more thereof. The hydroxypropyl cellulose (HPC) of different viscosity levels includes, for example, a low viscosity hydroxypropyl cellulose (HPC-L) having a viscosity of about 6 to about 10 mPa·s in a 2% aqueous solution at 20° C.; a medium viscosity hydroxypropyl cellulose (HPC-M) having a viscosity of about 150 to about 400 mPa·s in a 2% aqueous solution at 20° C., and a high viscosity hydroxypropyl cellulose (HPC-H) having a viscosity of about 1000 to about 4000 mPa·s in a 2% aqueous solution at 20° C. When hydroxypropyl cellulose (HPC) is used, then using a mixture of a medium viscosity hydroxypropyl cellulose (HPC-M) and a low viscosity hydroxypropyl cellulose (HPC-L) is preferable. In addition, the hydrophilic gel-forming high molecular substance in the core and in the outer shell may be either the same or different.

The outer shell contains a hydrophilic gel-forming high molecular substance in an amount of 76 to 95% by weight, preferably in an amount of 78 to 85% by weight, more preferably in an amount of 78 to 82% by weight, based on the total weight of the outer shell.

The disintegration suppression substance used in the outer shell of the present invention may be a substance made from pH-independent water-insoluble polymers which are usually used as bases or films for sustained release formulations. Said disintegration suppression substance may give an optimal strength to the outer shell, by which the tablet of the present invention achieves the stable disintegration under the circumstance of high mechanical irritation like in the digestive tract. By blending a disintegration suppression substance into the outer shell, the contents of a hydrophilic gel-forming high molecular substance or other excipients can be reduced without causing any change in the dissolution properties of nifedipine. As a result, the volume of the core can be miniaturized into about 70%.

In the outer shell, said disintegration suppression substance forms a matrix with the nifedipine and the hydrophilic gel-forming high molecular substance. The matrix means a condition where a pharmaceutically active ingredient is dispersed uniformly in a state of molecule or as fine particle in a base. Namely, the nifedipine of the present invention is uniformly dispersed in the hydrophilic gel-forming high molecular substance and the disintegration suppression substance.

The disintegration suppression substance to be blended in the outer shell includes, for example, the same ones as used in the shell of the press coated tablet disclosed in Patent Document 1, which can be used alone or a combination of two or more thereof. As to such disintegration suppression substance, specific pH-independent water-insoluble polymers which are usually used as bases or films for sustained release formulations can be exemplified, and for example, ethyl cellulose, or preferably water-insoluble methacrylate copolymers can be exemplified. Among them, ethyl acrylate•methyl methacrylate•trimethylammoniumethyl methacrylate chloride copolymer (hereafter referred to as “aminoalkyl methacrylate copolymer”) composed of the three recurring units as indicated by the following formulae (I), (II) and (III) is most preferred. Said “ethyl acrylate•methyl methacrylate•trimethylammoniumethyl methacrylate chloride copolymer” means either “Ammonio Methacrylate Copolymer Type A” ph. Eur. or “Ammonio Methacrylate Copolymer Type B” ph. Eur. in EP Official Compendium.

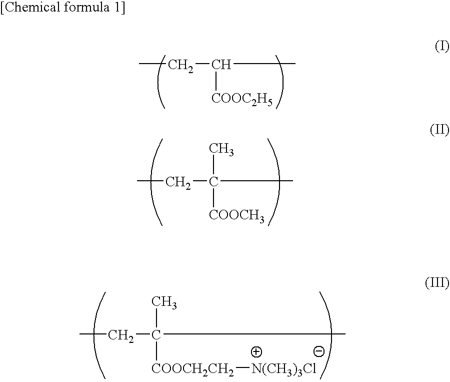

As to said aminoalkyl methacrylate copolymer, ones in which the weight ratio of the recurring units of above formulae (I), (II) and (III), i.e., the weight ratio of (I)/(II)/(III), is in the range 1/2/0.1-0.2 are preferred, or ones having a viscosity of from about 1 to about 4 centistokes are preferred. Such aminoalkyl methacrylate copolymers are commercially available under trade names of Eudragit RS or RL (manufactured by Roehm Pharma G.m.b.H., Germany).

As to the disintegration suppression substance contained in the outer shell, the combined contents of such a disintegration suppression substance and nifedipine contained in the outer shell can be kept in the range of 8-22% by weight, preferably in the range of 17-20% by weight, based on the total weight of the outer shell.

Namely, when the content of nifedipine in the outer shell is increased, then the content of a disintegration suppression substance in the outer shell is decreased. In the outer shell, the weight ratio of nifedipine content to disintegration suppression substance content is, for example, 2:1 to 3:1, or 1:1 to 1:4, but the present invention should not be construed to be limited thereto.

The disintegration suppression substance is contained in the outer shell in an amount of 2 to 20% by weight, preferably in an amount of 2.5 to 10% by weight, more preferably in an amount of 3 to 8% by weight, based on the total weight of the outer shell.

In order to promote the dissolution of nifedipine from the outer shell by improving the easiness of getting soaked of nifedipine crystals or by promoting the permeation of water or gastrointestinal duct fluid into the outer shell, a surfactant can be blended into the outer shell. The surfactant to be blended includes, for example, polysorbate 80, polysorbate 60, polysorbate 20, sodium lauryl sulfate, various sucrose fatty acid esters, etc., and these surfactants may be used alone or in a combination of two or more of these surfactants. The surfactant is normally contained in an amount of 0.03 to 3% by weight, preferably in an amount of 0.05 to 2% by weight, more preferably in an amount of 0.1 to 0.5% by weight, based on the total weight of the core.

Further, a fluidizing agent may optionally be blended in the outer shell, if necessary, and the fluidizing agent to be used is, for example, light anhydrous silicic acid, heavy anhydrous silicic acid, hydrated silicon dioxide, synthetic aluminum silicate, magnesium aluminometasilicate, etc. The content of these fluidizing agents is normally in the range of 0.02 to 2% by weight, preferably in the range of 0.05 to 1% by weight, more preferably in the range of 0.1 to 0.5% by weight, based on the total weight of the outer shell.

In addition, a lubricant may be added to the outer shell, if necessary. The lubricant to be used is, for example, magnesium stearate, stearic acid, sodium stearyl fumarate, sucrose fatty acid ester, talc, etc. The content of these lubricants is normally in the range of 0.02 to 2% by weight, preferably in the range of 0.05 to 1% by weight, more preferably in the range of 0.1 to 0.5% by weight, based on the total weight of the outer shell.

Further, the outer shell, if necessary, may additionally contain pharmaceutical additives which include excipients such as starches (e.g., corn starch, potato starch, alpha-starch, dextrin, carboxymethyl starch, etc.), sugars (e.g., lactose, sucrose, glucose, mannitol, sorbitol, etc.), inorganic salts (e.g., light anhydrous silicic acid, synthetic aluminum silicate, magnesium aluminometasilicate, calcium phosphate, calcium carbonate, etc.), oils and fats (e.g., paraffin, wax, higher fatty acid, etc.), and cellulosic substances; disintegrating agents such as starches, croscarmellose sodium, sodium carboxymethyl starch, carboxymethylcellulose, carboxymethycellulose calcium, low substituted hydroxypropylcellulose, crystalline cellulose, crosslinked polyvinylpyrrolidone, etc.; coloring agents (pigments) such as various food colors, iron sesquioxide, etc; etc.

However, in order to miniaturize the outer shell, it is desirable to suppress the contents of these pharmaceutical additives as low as possible.

The final composition of the outer shell is preferably adjusted so that the dissolution rate of nifedipine from a tablet prepared by using only the composition of said outer shell is

in the dissolution test using a sinker according to Method 2 of the dissolution test method as prescribed by The Japanese Pharmacopoeia [hereafter referred to as “Pharmacopoeia Method 2 dissolution test (using sinker)”] is,

after 2 hours, 20 to 50%, preferably 25 to 45%, more preferably 30 to 40%;

after 4 hours, 40 to 90%, preferably 45 to 85%, more preferably 50 to 80%.

The tablet used for the dissolution test can be either one with film coating for light protection or one without film coating for light protection.

In the present specification, Pharmacopoeia Method 2 dissolution test (using sinker) is conducted under the following conditions:
Test liquid: Phosphate buffer solution at pH 6.8, containing 1% of sodium lauryl sulfate, 900 ml
Temperature: 37° C.
Rotation rate: 100 rpm
Fixed position of the sinker:
   Fixed at the center between the test liquid level and the upper edge of the stirring blade, and spaced from the container wall by 10 mm, with acid-resistant wire of the diameter of not more than 1.0 mm.

The dissolution test according to the disintegration test method as prescribed by the Japanese Pharmacopoeia (Pharmacopoeia Disintegration test device method) is conducted under the following conditions:
Test liquid: Phosphate buffer solution at pH 6.8, containing 0.5% of polysorbate 80, 900 ml
Temperature: 37° C.
An auxiliary disc is used.

On the other hand, the core contains nifedipine and a hydrophilic gel-forming high molecular substance as essential components like the outer shell does, and if necessary, further may contain similar surfactants, fluidizers, lubricants and other pharmaceutical additives to those of the outer shell.

Nifedipine, hydrophilic gel-forming high molecular substances, surfactants, fluidizers, lubricant and other pharmaceutical additives to be used in the core which are similar to those of the outer shell may be preferably selected from ones as listed in the above which are used in the outer shell, and the selected ones may be the same or different.

In the core, in order to promote the dissolution of nifedipine by improving the easiness of getting soaked of nifedipine crystals or by promoting the permeation of water or gastrointestinal duct fluid into the inner of the tablet, the core may additionally contain a surfactant. The content of said surfactant is usually in the range of 0.02 to 2% by weight, preferably in the range of 0.05 to 1% by weight, more preferably in the range of 0.1 to 0.5% by weight, based on the total weight of the core.

When the core contains a fluidizing agent, the content thereof is usually in the range of 0.02 to 2% by weight, preferably in the range of 0.05 to 1% by weight, more preferably in the range of 0.1 to 0.5% by weight, based on the total weight of the core.

When the core contains a lubricant agent, the content thereof is usually in the range of 0.02 to 2% by weight, preferably in the range of 0.05 to 1% by weight, more preferably in the range of 0.1 to 0.5% by weight, based on the total weight of the core.

Further, the core may contain, in addition to the above components, the same various pharmaceutical additives as those of the outer shell, if necessary.

The final composition of the core is usually adjusted preferably so that the dissolution rate of nifedipine from a tablet having the same composition as that of the core, as tested by the dissolution test according to Method 2 of the dissolution test method but not using a sinker as prescribed by The Japanese Pharmacopoeia [hereafter referred to as “Pharmacopoeia Method 2 dissolution test (not using sinker)”] is:

after 45 minutes, 20-70%, preferably 25-65%, more preferably 30-60%:

after 2 hours, at least 65%, preferably at least 70%, more preferably at least 75%.

The tablet used in the dissolution test can be either one with film coating for light protection or one without film coating for light protection.

Pharmacopoeia Method 2 dissolution test (not using sinker) is conducted under the following conditions:
Test liquid: Phosphate buffer solution at pH 6.8, containing 1% of sodium lauryl sulfate 900 ml
Temperature: 37° C.
Rotation rate: 75 rpm.

The tablet having the same composition as that of the core to be used in the above dissolution test is compressed under the same conditions employed for compressing the core of the press coated tablet of the present invention.

The press coated tablet comprising the core and the outer shell as mentioned above may be prepared by, for example, forming a nucleus tablet to serve as the core by a known means, and then coating said nucleus tablet with the outer shell having the above-described composition, using a press coating machine (press coater). The compressing conditions in that occasion are not strictly limited, but are variable depending on the desired dissolution properties, etc., of the final tablets. Usually, suitable compressing pressure for the nucleus tablet (core) is in the range of about 100-1000 kgf, and that for the press coated tablets is in the range of about 500-2000 kgf.

According to the present invention, the diameter of the core can be generally within the range of 4.5-5.5 mm, preferably 4.5 to 5.2 mm, more preferably 4.6 to 5.1 mm, and the thickness of the core can be within the range of 1.5 to 2.5 mm, preferably 1.7 to 2.3 mm, more preferably 1.8 to 2.2 mm. Further, the diameter of the press coated tablet prepared by press-coating (tableting) said core with an outer shell can be usually within the range of 7.5 to 8.5 mm, preferably 7.7 to 8.3 mm, more preferably 7.8 to 8.2 mm, and the thickness thereof can be within the range of 4.5 to 5.2 mm, preferably 4.5 to 5.1 mm, more preferably 4.6 to 5.0 mm.

The above-mentioned core may be applied with a thin film coating prior to being coated with the outer shell. As the base for such film coating, cellulosic water-soluble coating bases such as HPC, HPMC, hydroxyethyl cellulose, methylhydroxyethyl cellulose, etc.; cellulosic enteric coating bases such as hydroxypropylmethyl cellulose phthalate, hydroxypropylmethyl cellulose acetate succinate, carboxymethylethyl cellulose, cellulose acetate phthalate, etc.; and other enteric film coating bases such as methacrylic acid copolymers, shellac, etc.; and the like may be exemplified.

Furthermore, the press coated tablet thus obtained as above may be applied with at least one layer of film coating for light protection on the surface of said tablet after forming the press coated tablet. As such film coating for light protection, for example, cellulosic water-soluble coating blended with an adequate amount of a light-shielding agent, e.g., iron sesquioxide and/or titanium dioxide, may be exemplified. Among them, HPMC is particularly preferred as the cellulosic water-soluble coating because of its good film-forming property, and HPMC whose 2% aqueous solution has a viscosity not higher than 100 mPa·s, in particular, not higher than 15 mPa·s at 20° C. is especially preferred. These film coating bases may contain a plasticizing agent, if necessary, such as polyethylene glycol, etc.

Thus prepared nifedipine-containing press coated tablets of the present invention can exhibit the same nifedipine dissolution properties as those of the once-a-day dosed nifedipine-containing press coated tablet as disclosed in Patent Document 1, even though it is miniaturized. Namely, the dissolution rate of nifedipine from the miniaturized nifedipine-containing press coated tablet of the present invention is:

(a) in the dissolution test using a sinker according to Method 2 of the dissolution test method as prescribed by The Japanese Pharmacopoeia [hereafter referred to as “Pharmacopoeia Method 2 dissolution test (using sinker)”] is,

after 2 hours, 10 to 40%, preferably 15 to 35%, more preferably 20 to 29%;

after 4 hours, 30 to 65%, preferably 35 to 60%, more preferably 39 to 56%;

after 6 hours, at least 55%, preferably at least 60%, more preferably at least 65%, and

(b) in the dissolution test according to the disintegration test method as prescribed by The Japanese Pharmacopoeia [hereafter referred to as “Pharmacopoeia Disintegration test device method”] is,

after 3 hours, 35 to 65%, preferably 40 to 60%, more preferably 40 to 50%;

after 4 hours, at least 65%, preferably 65 to 90%)/0, more preferably 70 to 80%.

The nifedipine-containing press coated tablet used for (a) the dissolution test using a sinker according to Method 2 of the dissolution test method as described by The Japanese Pharmacopoeia and (b) the dissolution test according to the disintegration test method as prescribed by The Japanese Pharmacopoeia can be either one having at least one layer of film coating for light protection being applied on the surface of said tablet, or one having no film coating for light protection.

The nifedipine-containing press coated tablet of the present invention can keep the effective blood concentration of nifedipine for a long time (at least for 24 hours), and further since it is miniaturized, it is easier for patients to swallow or take it as compared to the conventional commercially available tablets.

In addition, since the nifedipine-containing press coated tablet provided by the present invention can be miniaturized as mentioned above, it is possible to prepare a combination drug of nifedipine and a second pharmaceutically active ingredient by setting a rapidly-dissolving part containing said second pharmaceutically active ingredient onto said nifedipine-containing press coated tablet. Thus, the present invention additionally provides a tablet which is characterized in that a rapidly-dissolving part containing a second pharmaceutically active ingredient partly coats compressively onto said nifedipine-containing press coated tablet in such a manner that at least a part of the surface thereof is exposed (hereinafter, referred to as the combination drug).

The combination drug of the present invention is a tablet which consists of a miniaturized nifedipine-containing press coated tablet of the present invention and a rapidly-dissolving part containing a second pharmaceutically active ingredient, and where said rapidly-dissolving part is applied in such a manner that at least one face among three faces of the upper face, the bottom face and the side face of said nifedipine-containing press coated tablet is externally exposed.

Such tablets include, for example, a simple parallel bi-layer or three-layer coated tablet where a rapidly-dissolving part coats compressively on one of the upper face or the bottom face, or both of these faces of the above mentioned miniaturized nifedipine-containing press coated tablet, or a pressed tablet where the bottom face or side face of the above mentioned miniaturized nifedipine-containing press coated tablet is enveloped with a rapidly-dissolving part containing a second pharmaceutically active ingredient, and the upper face of said nifedipine-containing press coated tablet is substantially exposed concentrically to the upper face of said rapidly-dissolving part (another name; called as bull's eye tablet). Although it is possible to compressively coat all of the faces of said miniaturized nifedipine-containing press coated tablet with a rapidly-dissolving part containing a second pharmaceutically active ingredient to give a press coated three-layer tablet, the final tablet thus obtained becomes too large so that it may possibly hamper convenience of patients.

The second pharmaceutically active ingredient to be used in the combination drug of the present invention may be any ones which do not exhibit any adverse effect by interacting with nifedipine, and require the rapidly-dissolving properties, and further for the purpose of once-a-day oral administration, and preferably ones showing synergistic pharmaceutical effects when administered together with nifedipine.

Such second pharmaceutically active ingredients are, for example, angiotensin II receptor antagonists selected from the group consisting of candesartan cilexetil, loxartan potassium, valsartan, olmesartan medoxomil, telmisartan and irbesartan; and angiotensin converting enzyme inhibitors selected from the group consisting of enalapril maleate, lisinopril, cilazapril and delapril hydrochloride. Among them, candesartan cilexetil and enalapril maleate are especially preferable for a combination drug with the miniaturized nifedipine-containing press coated tablet of the present invention because the dosages of these active ingredients are small so that the volume of the rapidly-dissolving part can be reduced.

In addition, diuretics can also be usable as a second pharmaceutically active ingredient which have been used from the old days as an antihypertensive agent exhibiting a moderate antihypertensive effect. Diuretics being able to be used in this purpose include, for example, thiazide diuretics such as ethiazide, cyclopenthiazide, trichloromethiazide, hydrochlorothiazide, hydroflumethiazide, benzylhydrochlorothiazide, polythiazide, methychlothiazide, penflutizide, chlorothiazide, cyclothiazide, flumethiazide, benzthiazide, bendroflumethiazide, etc.; non-thiazide diuretics such as acetazolamide, azosemide, etacrynic acid, sodium etacrynate, quinethazone, clofen-amide, chlorthalidone, spironolactone, triamterene, piretanide, bumetanide, furosemide, metolazone, mefruside, amiloride, indapamide, ticrynafen, tripamide, torsemide, meticrane, aminophylline, etc.

The content of the second pharmaceutically active ingredient to be used in the combination drug of the present invention may vary depending on the usage, dosage, formability, bulkiness of said second pharmaceutically active ingredient, but it is usually not more than 150 mg, preferably not more than 120 mg, more preferably not more than 100 mg, and further preferably not more than 80 mg.

The rapidly-dissolving part containing said second pharmaceutically active ingredient may further contain necessary pharmaceutical additives such as disintegrators, binders, surfactants, lubricants, etc. These pharmaceutical additives may the same ones as used in the above-mentioned miniaturized nifedipine-containing press coated tablet. The characteristics of the rapidly-dissolving part are to release the second pharmaceutically active ingredient into the stomach by disintegrating in a short time after the administration. For this purpose, necessary pharmaceutical additives such disintegrators, binders, surfactants, lubricants, etc. should be adequately selected or combined, by which the composition of rapidly-dissolving part is determined. The content rate of said second pharmaceutically active ingredient in the rapidly-dissolving part can be determined by taking into consideration the dosage, physiochemical properties, especially solubility or easiness of getting soaked of second pharmaceutically active ingredient.

The dissolution rate of said second pharmaceutically active ingredient from the rapidly-dissolving part is preferably designed in such a manner that the dissolution rate thereof is at least 80% after one hour in the dissolution test (50 rpm) according to Method 2 of the dissolution test method as prescribed by The Japanese Pharmacopoeia. In this specification, the dissolution test (50 rpm) according to Method 2 of the dissolution test method as prescribed by the Japanese Pharmacopoeia is conducted under the following conditions.
Test liquid: Purified water containing 0.5% of polysorbate 80, 900 ml
Temperature: 37° C.
Rotation rate: 50 rpm.

The miniaturized nifedipine-containing press coated tablet of the present invention and the rapidly-dissolving part containing said second pharmaceutically active ingredient may be bonded by using the same or similar press coating machine (press coater) to that is used in the preparation of the above-mentioned miniaturized nifedipine-containing press coated tablet. A commercially available machine can be used as it stands, but may be used after remodeling. The tableting is conducted, for example, by one of the following three methods, which is suitably selected due to the capability of a press coating machine (press coater) to be used.

(1) Method where a press coating machine (press coater), which has a mortar and pestle with the same diameter or a little larger diameter (e.g., larger by about 0.05 to 0.2 mm) than that of the nifedipine-containing press coated tablet of the present invention, is used, and a powder to be tableted for the rapidly-dissolving part is filled into the mortar thereof, and further a nifedipine-containing press coated tablet is placed onto it, and then the mixture is compressively formulated within the mortar without any further filling.

In this method, the rapidly-dissolving part containing the second pharmaceutically active ingredient is bonded in laminae only on the bottom face of the nifedipine-containing press coated tablet, and in the obtained tablet, the side face and the upper face of the nifedipine-containing press coated tablet are externally exposed. Therefore, this method is especially useful for the cases that the dosage of the second pharmaceutically active ingredient is small enough, for example, 10 mg or less, by which the rapidly-dissolving part can be designed to be small.

(2) Method where a press coating machine (press coater), which has a mortar and pestle with the same diameter or a little larger diameter (e.g., larger by about 0.05 to 0.2 mm) than that of the nifedipine-containing press coated tablet of the present invention, is used, and a powder to be tableted for the rapidly-dissolving part is filled into the mortar thereof, and further a nifedipine-containing press coated tablet is placed onto it, and then further a powder to be tableted for the rapidly-dissolving part is filled, then the mixture is compressively formulated within the mortar.

By this method, the rapidly-dissolving part containing the second pharmaceutically active ingredient is bonded on both of the upper face and the bottom face of the nifedipine-containing press coated tablet in laminae, and in the obtained tablet, the side face of the nifedipine-containing press coated tablet is only externally exposed. Therefore, this method is especially useful for the cases that the dosage of the second pharmaceutically active ingredient is small enough, for example, 10 mg or less, by which the rapidly-dissolving part can be designed to be small.

(3) Method where a press coating machine (press coater), which has a mortar and pestle with a larger diameter than that of the nifedipine-containing press coated tablet of the present invention (usually the diameter is larger by 2.4 mm or more, but the figure thereof may be either round shape or oval shape) is used, and a powder to be tableted for the rapidly-dissolving part is filled into the mortar thereof, and a nifedipine-containing press coated tablet is placed onto it, and then the mixture is compressively formulated within the mortar without any further filling.

By this method, the bottom face and the side face of the nifedipine-containing press coated tablet are bonded with the rapidly-dissolving part containing the second pharmaceutically active ingredient in concavity, by which there is obtained a tablet where substantially only the upper face of the nifedipine-containing press coated tablet is exposed concentrically to the upper face of the rapidly-dissolving part. By this method, a combination drug can be freely designed because of few constraint from the dosage of the second pharmaceutically active ingredient so that more types of second pharmaceutically active ingredients can be employed. Therefore, this method can be most wildly usable method.

In any of the above Methods (1) to (3), in order to obtain a good bond between the nifedipine-containing press coated tablet and the rapidly-dissolving part containing a second pharmaceutically active ingredient, it is preferable to avoid the film-coating of the nifedipine-containing press coated tablet in advance.

The tableting pressure for press coating is controlled into within a range where the nifedipine-containing press coated tablet is substantially not destroyed, but the rapidly-dissolving part can show a suitable hardness and disintegrative properties. Usually, the tableting pressure for press coating is in the range of about 500 to 2000 kgf.

Further, when the size of the final tablet is large, it may become difficult for the aged patients to swallow, so that it is desirable to design the size of the tablet for patients to easily swallow. When the tablet is in a round shape, the diameter of the final tablet is usually not more than 12 mm, preferably not more than 10 mm, more preferably not more than 9.5 mm, and the thickness of the tablet is usually not more than 6 mm, preferably not more than 5.8 mm, more preferably not more than 5.6 mm. When it is not evitable to make the rapidly-dissolving part large, then the shape of the tablet is preferably designed in an oblong shape or oval shape, and the minor axes thereof should be designed 9 mm or about.

The combination drug consisting of the miniaturized nifedipine-containing press coated tablet of the present invention and the rapidly-dissolving part containing a second pharmaceutically active ingredient may optionally be subjected to conventional film-coating, if necessary. The composition of the film-coating is the same as that to be used in the above-mentioned miniaturized nifedipine-containing press coated tablet.

When the combination drug consisting of the nifedipine-containing press coated tablet and the rapidly-dissolving part containing a second pharmaceutically active ingredient prepared as mentioned above is administered orally to human being, then the rapidly-dissolving part is rapidly disintegrated to release the second pharmaceutically active ingredient. Subsequently, nifedipine is dissolved out stably from the nifedipine-containing press coated tablet, by which the effective blood concentration can be kept for over a period of 24 hours.

### EXAMPLES

Hereafter, a preferred embodiment of the present invention is more specifically explained by Examples and Comparative Examples. However, the present invention should not be construed to be limited thereto. In addition, HPC-L and HPC-M which were used in Examples and Comparative Examples are respectively hydroxypropylcellulose products manufactured by Nippon Soda K.K., having a viscosity of 6.0-10.0 mPa·s (2% aqueous solution, 20° C.), and hydroxypropylcellulose products manufactured by Nippon Soda K.K., having a viscosity of 150-400 mPa·s (2% aqueous solution, 20° C.). As HPMC, hydroxypropyl methylcellulose 2910 manufactured by Shin-Etsu Kagaku Kogyo K.K., having a viscosity of 15 mPa·s was used. “Eudragit RL PO” is a commercial name of a product of Roehm Pharma GmbH of Germany, for ethyl acrylate•methyl methacrylate•trimethylammoniumethyl methacrylate chloride copolymer.

### Example 1

| | |
|---|---|
| Nifedipine (micropulverized crystals) | 180 g (30%) |
| Lactose | 271 g (45%) |
| HPC-L | 146 g (24%) |

The above starting materials were homogeneously mixed, granulated, dried and sieved, to which 2 g of magnesium stearate was added and mixed. The mixture was tableted under a tableting pressure of 0.5 ton with a rotary press tableting machine (Kikusui), to give the core tablets weighing 50 mg per tablet (diameter: 5 mm, thickness: 2 mm).

| | |
|---|---|
| Nifedipine (micropulverized crystals) | 100 g (14%) |
| HPC-L | 388 g (54%) |
| HPC-M | 192 g (27%) |
| Eudragit RLPO | 36 g (5%) |

The above starting materials were homogeneously mixed, granulated, dried and sieved. Then 4 g of magnesium stearate was added thereto and mixed. The resulting composition was made the outer shell which, together with the previously prepared core tablet, was tableted under a tableting pressure of 0.8 ton with a press coating tableting machine (Kikusui Cleanpress Correct 18DC), to give press coated tablets containing 40 mg of nifedipine and weighing 230 mg per tablet (diameter: 8 mm, thickness: 4.8 mm). Further, said press coated tablets were applied with film coating for light protection having the following composition on the surface to give tablets having a total weight of 237 mg, and a volume of about 215 mm³ (this volume is about 70% of that of the commercially available tablets).

Composition of film coating per tablet:

| | |
|---|---|
| HPMC 2910 (15 mPa · s) | 4.2 mg (60%) |
| Polyethylene glycol 4000 | 1.4 mg (20%) |
| Iron sesquioxide (red) | 0.3 mg (4%) |
| Titanium dioxide | 1.1 mg (16%) |

### Comparative Example 1

| | |
|---|---|
| Nifedipine (micropulverized crystals) | 100 g (14%) |
| HPC-L | 352 g (49%) |
| HPC-M | 192 g (27%) |
| Eudragit RLPO | 72 g (10%) |

The above starting materials were homogeneously mixed, granulated, dried and sieved, to which 4 g of magnesium stearate was added and mixed. The resulting composition was made the outer shell which, together with the previously prepared core tablet obtained in Example 1, was tableted under a tableting pressure of 0.8 ton with a press coating tableting machine (Kikusui Cleanpress Correct 18DC), to give press coated tablets containing 40 mg of nifedipine and weighing 230 mg per tablet (diameter: 8 mm, thickness: 4.8 mm). The press coated tablets were further applied with film coating for light protection in a similar manner to Example 1 on the surface to give tablets having a total weight of 237 mg, and the volume of about 215 mm³ (this volume is about 70% of that of the commercially available tablets).

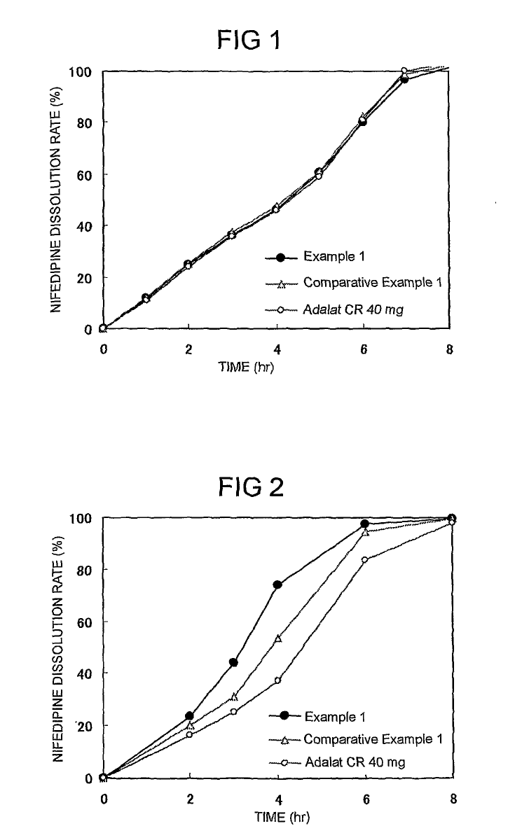

As to the tablets obtained in the above Example 1 and Comparative Example 1, and the commercially available Adalat CR 40 mg, the nifedipine dissolution rate therefrom was measured according to Pharmacopoeia Method 2 dissolution test (using sinker) and Pharmacopoeia Disintegration test device method under the above-mentioned conditions. The dissolution patterns of these tablets are shown in FIG. 1 and FIG. 2.

### Example 2

| | |
|---|---|
| Nifedipine (micropulverized crystals) | 45 g (8%) |
| Lactose | 229 g (38%) |
| HPC-L | 323 g (54%) |

The above starting materials were homogeneously mixed, granulated, dried and sieved, to which 2 g of magnesium stearate was added and mixed. The mixture was tableted under a tableting pressure of 0.5 ton with a rotary press tableting machine (Kikusui), to give the core tablets weighing 50 mg per tablet (diameter: 5 mm, thickness: 2 mm).

| | |
|---|---|
| Nifedipine (micropulverized crystals) | 38 g (4%) |
| HPC-L | 563 g (52%) |
| HPC-M | 270 g (25%) |
| Ethyl cellulose | 102 g (9%) |
| Crystalline cellulose | 102 g (9%) |

The above starting materials were homogeneously mixed, granulated, dried and sieved. Then 6 g of magnesium stearate was added thereto and mixed. The resulting composition was made the outer shell which was tableted together with the previously prepared core tablet under a tableting pressure of 0.8 ton with a press coating tableting machine (Kikusui Cleanpress Correct 18DC), to give press coated tablets containing 10 mg of nifedipine and weighing 230 mg per tablet (diameter: 8 mm, thickness: 4.8 mm, total weight: 230 mg, volume: about 215 mm³ (this volume is about 70% of that of the commercially available tablets)). Further, if desired, said press coated tablets can be applied with film coating for light protection of the following composition on the surface.

Composition of film coating per tablet:

| | |
|---|---|
| HPMC 2910 (15 mPa · s) | 3.7 mg (53%) |
| Polyethylene glycol 4000 | 1.2 mg (18%) |
| Iron sesquioxide (red) | 0.9 mg (12%) |
| Titanium dioxide | 1.2 mg (17%) |

### Example 3

| | |
|---|---|
| Nifedipine (micropulverized crystals) | 38 g (4%) |
| HPC-L | 563 g (52%) |
| HPC-M | 270 g (25%) |
| Ethyl cellulose | 102 g (9%) |
| Calcium phosphate | 102 g (9%) |

The above starting materials were homogeneously mixed, granulated, dried and sieved. Then 6 g of magnesium stearate was added thereto and mixed. The resulting composition was made the outer shell which was tableted together with the previously prepared core tablet under a tableting pressure of 0.8 ton with a press coating tableting machine (Kikusui Cleanpress Correct 18DC), to give press coated tablets containing 10 mg of nifedipine and weighing 230 mg per tablet (diameter: 8 mm, thickness: 4.8 mm, total weight: 230 mg, volume: about 215 mm³ (this volume is about 70% of that of the commercially available tablets)). Further, if desired, said press coated tablets can be applied with film coating for light protection on the surface in a similar manner to Example 2.

### Comparative Example 2

| | |
|---|---|
| Nifedipine (micropulverized crystals) | 38 g (4%) |
| HPC-L | 677 g (63%) |
| HPC-M | 324 g (30%) |
| Ethyl cellulose | 36 g (3%) |

The above starting materials were homogeneously mixed, granulated, dried and sieved, to which 6 g of magnesium stearate was added and mixed. The resulting composition was made the outer shell which was tableted together with the previously prepared core tablet obtained in Example 2 under a tableting pressure of 0.8 ton with a press coating tableting machine (Kikusui Cleanpress Correct 18DC), to give press coated tablets containing 10 mg of nifedipine and weighing 230 mg per tablet (diameter: 8 mm, thickness: 4.8 mm, total weight: 230 mg, volume: 215 mm³ (this volume is about 70% of that of the commercially available tablets)). If desired, the press coated tablets can be further applied with film coating for light protection on the surface in a similar manner to Example 2.

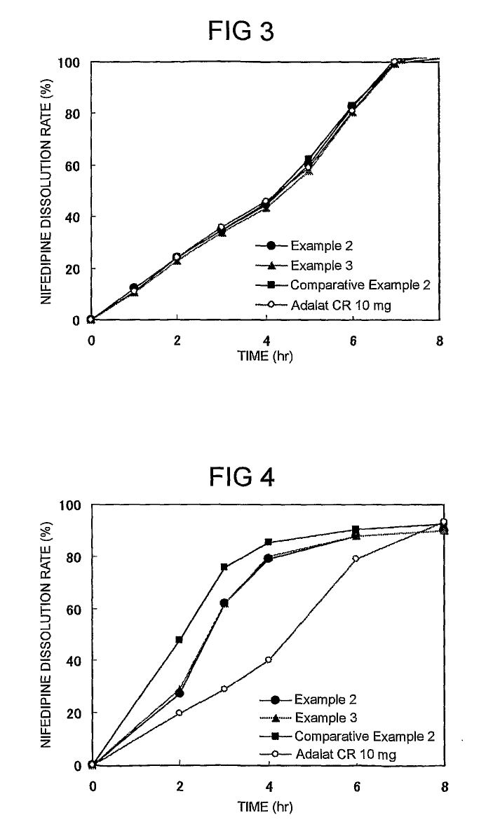

As to the tablets (which were not applied with film coating for light protection) obtained in the above Example 2, Example 3 and Comparative Example 2 and the commercially available Adalat CR 10 mg, the nifedipine dissolution rate therefrom was measured in a similar manner to Comparative Example 1, etc. according to Pharmacopoeia Method 2 dissolution test (using sinker) and Pharmacopoeia Disintegration test device method. The dissolution patterns of these tablets are shown in FIG. 3 and FIG. 4.

### Experiment 1

The pharmacokinetic parameters were compared when each one tablet obtained in the above Example 1 and Comparative Example 1 and the commercially available once-a-day administration type sustained release nifedipine tablet (trade name: Adalat CR 40 mg, containing 40 mg of nifedipine) were orally administered once after eating to 12 healthy adult males. The results are shown in Table 1.

TABLE 1
Pharmacokinetic parameters

| | Maximum Plasma | Area under the blood |
|---|---|---|
| | Concentration | concentration time curve |
| Test Tablet | C max (μg/L) | AUC₀₋ₜₙ (hr × μg/L) |
| Example 1 | 72 | 572 |
| | Point estimate: 92% | Point estimate: 93% |
| Comparative | 65 | 546 |
| Example 1 | Point estimate: 87% | Point Estimate: 84% |
| Adalat CR 40 mg | 77 | 626 |

As is apparent from the above results, the tablet of Example 1 of the present invention showed the equivalent maximum plasma concentration and the area under the blood concentration time curve to those of the commercially available once-a-day administration type sustained release nifedipine tablet. Accordingly, it can be considered that the tablets of Examples show an equal transitional change in the nifedipine plasma concentration to that of the commercially available once-a-day administration type sustained release nifedipine tablet.

On the other hand, the tablet of Comparative Example 1 showed a lower maximum plasma concentration and a lower area under the blood concentration time curve than those of the commercially available once-a-day administration type sustained release nifedipine tablet, and hence, it could not be said that they are equal.

### INDUSTRIAL APPLICABILITY

The nifedipine-containing press coated tablet of the present invention is miniaturized as compared with the nifedipine-containing tablets being currently used, so that it is easy for patients to take the nifedipine-containing press coated tablet of the present invention. In addition, the dissolution properties and bioavailability of nifedipine are not changed in the tablet of the present invention as compared to the currently-used nifedipine-containing tablets, so that the tablet of the present invention can release nifedipine sustainably, and hence, the tablet of the present invention is quite useful as a sustained release nifedipine-containing tablet.

1. A nifedipine-containing press coated tablet which comprises
(A) a core comprising nifedipine and a hydrophilic gel-forming high molecular substance, wherein the dissolution of the nifedipine from the core is designed to be delayed: and
(B) an outer shell which compressively coats the above core and which comprises
(i)(a) nifedipine and (b) a disintegration suppression substance comprising a pH-independent water-insoluble polymer, wherein the combined contents of (a) and (b) are in the range of 8 to 22% by weight based on the total weight of the outer shell, and
(ii) a hydrophilic gel-forming high molecular substance selected from cellulose derivatives and polyvinyl alcohols, wherein the content thereof is in the range of 76 to 95% by weight based on the total weight of the outer shell,
which is characterized in that in the outer shell, the disintegration suppression substance forms a matrix with nifedipine and a hydrophilic gel-forming high molecular substance, and the diameter of the nifedipine-containing press coated tablet is 7.5 to 8.5 mm, and the thickness thereof is 4.5 to 5.2 mm,
and further characterized in that the dissolution rate of the nifedipine from said tablet is:
(a) in the dissolution test using a sinker according to Method 2 of the dissolution test method as prescribed by The Japanese Pharmacopoeia,
10-40% after 2 hours;
30-65% after 4 hours;
at least 55% after 6 hours, and
(b) in the dissolution test according to the disintegration test method as prescribed by The Japanese Pharmacopoeia,
35-65% after 3 hours;
at least 65% after 4 hours.

2. The nifedipine-containing press coated tablet according to claim 1, wherein the disintegration suppression substance is ethyl cellulose or ethyl acrylate•methyl methacrylate•trimethylammoniumethyl methacrylate chloride copolymer.

3. The nifedipine-containing press coated tablet according to claim 1, wherein the disintegration suppression substance is ethyl acrylate•methyl methacrylate•trimethylammoniumethyl methacrylate chloride copolymer.

4. The nifedipine-containing press coated tablet according to claim 1, wherein the hydrophilic gel-forming high molecular substance in the core and the outer shell is hydroxypropyl cellulose or hydroxypropylmethyl cellulose.

5. The nifedipine-containing press coated tablet according to claim 1, wherein the hydrophilic gel-forming high molecular substance in the outer shell is a combination of a medium viscosity hydroxypropyl cellulose and a low viscosity hydroxypropyl cellulose.

6. The nifedipine-containing press coated tablet according to claim 1, wherein the content ratio of the nifedipine and the disintegration suppression substance in the outer shell is 2:1 to 3:1.

7. The nifedipine-containing press coated tablet according to claim 1, wherein the content ratio of the nifedipine and the disintegration suppression substance in the outer shell is 1:1 to 1:4.

8. The nifedipine-containing press coated tablet according to claim 1, wherein the content of nifedipine in the outer shell is 3 to 15% by weight based on the total weight of the outer shell.

9. The nifedipine-containing press coated tablet according to claim 1, wherein the combined content of the nifedipine and the disintegration suppression substance in the outer shell is 18 to 21% by weight based on the total weight of the outer shell.

10. The nifedipine-containing press coated tablet according to claim 1, wherein the dissolution rate of the nifedipine from said tablet is:
40 to 60% after 3 hours;
65 to 90% after 4 hours,
in the dissolution test according to the disintegration test method as prescribed by The Japanese Pharmacopoeia.

11. The nifedipine-containing press coated tablet according to claim 1, wherein the diameter of the core is 4.5 to 5.5 mm, and the thickness thereof is 1.5 to 2.5 mm.

12. A tablet, which is characterized by being prepared by partially compressively coating a nifedipine-containing press coated tablet as set forth in claim 1 with a rapidly-dissolving part comprising a second pharmaceutically active ingredient in such a manner that at least a part of the surface of said press coated tablet is exposed.

13. A method of preparing a nifedipine-containing press coated tablet, which comprises compressively coating (A) a core comprising nifedipine and a hydrophilic gel-forming high molecular substance, wherein the dissolution of the nifedipine from the core is designed to be delayed, with (B) an outer shell comprising (i) (a) nifedipine and (b) a disintegration suppression substance comprising a pH-independent water-insoluble polymer, wherein the combined contents of (a) and (b) are in the range of 8 to 22% by weight based on the total weight of the outer shell, and (ii) a hydrophilic gel-forming high molecular substance selected from cellulose derivatives and polyvinyl alcohols, wherein the content thereof is in the range of 76 to 95% by weight based on the total weight of the outer shell,
said tablet being characterized in that in the outer shell, the disintegration suppression substance forms a matrix with nifedipine and a hydrophilic gel-forming high molecular substance, and the diameter of the nifedipine-containing press coated tablet is 7.5 to 8.5 mm, and the thickness thereof is 4.5 to 5.2 mm,
and further characterized in that the dissolution rate of the nifedipine from said tablet is:
(a) in the dissolution test using a sinker according to Method 2 of the dissolution test method as prescribed by The Japanese Pharmacopoeia,
10-40% after 2 hours;
30-65% after 4 hours;
at least 55% after 6 hours, and
(b) in the dissolution test according to the disintegration test method as prescribed by The Japanese Pharmacopoeia,
35-65% after 3 hours;
at least 65% after 4 hours.

14. The method of preparing the nifedipine-containing press coated tablet according to claim 13, wherein the diameter of the core is 4.5 to 5.5 mm, and the thickness thereof is 1.5 to 2.5 mm.

15. A film-coating tablet, which comprises the tablet according to claim 1, and at least one layer of film coating for light protection being applied thereon.

16. A method of preparing a film-coating tablet, which comprises formulating a nifedipine-containing press coated tablet by the method according to claim 13, followed by applying at least one layer of film coating for light protection on the surface of said tablet.
